(19) United States
(12) Patent Application Publication
(10) Pub. No.: US 2018/0111938 A1
(43) Pub. Date: Apr. 26, 2018

(54) Synthesis of Intermediates Used in the Manufacture of Anti-HIV Agents

(71) Applicant: CBZ INVESTMENTS LTD., Essex (GB)

(72) Inventors: Braja Sundar PRADHAN, Telangana (IN); Kommireddy AMARNATH, Hyderabad (IN); Naresh VEMPALA, Hyderabad (IN); Md. Ataur RAHMAN, Hyderabad (IN)

(21) Appl. No.: 15/572,145

(22) PCT Filed: May 4, 2016
(86) PCT No.: PCT/IB2016/052548

§ 371 Date: Nov. 6, 2017

(30) Foreign Application Priority Data
May 5, 2015 (IN) 1786/MUM/2015

(51) Int. Cl. C07D 473/34; C07F 9/6561

(52) U.S. Cl. CPC C07D 473/34; C07F 9/65616

(57) ABSTRACT

The present invention relates to a process of preparing intermediates of Formula (I). The process comprises of reacting compound of Formula (III) with compound of Formula (V) in the presence of a solvent selected from an alcohol, ether or water to form compound of Formula (I) wherein, R¹ is selected from —NH₂, Cl, Br, NHCOR″, wherein R″ is alkyl, aryl, Schiff's base of formula N═CHR′, wherein R′ is alkyl or aryl; R² is selected from H, alkyl; R³ and R⁴, each independently is H; R⁵ and R⁶, each independently is H, alkyl; R⁷ is H, alkyl; and R⁸ is H, alkyl.

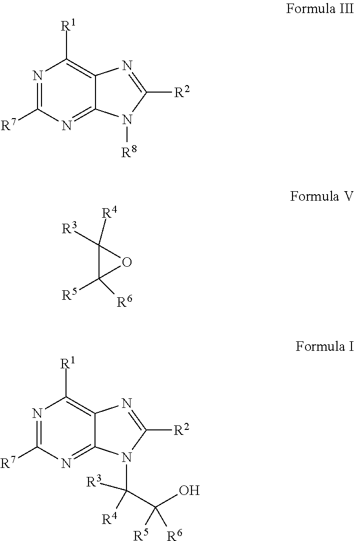

### FIELD OF THE INVENTION

The present invention relates to process for synthesis of intermediates used in the manufacture of anti-HIV drugs.

### BACKGROUND OF THE INVENTION

The U.S. Pat. No. 5,935,946A discloses a method for preparation of 9-[2-(R)-(hydroxyl)propyl]adenine. The method involved coupling (R)-1,2-propylene carbonate with adenine under basic condition in N,N-dimethylformamide (DMF) at 130° C. for 18-30 h to furnish the said product. The referred intermediate compound, (R)-1, 2-propylene carbonate in turn was prepared in two steps from (S)-Glycidol. In the first step, (S)-Glycidol was subjected to reductive hydrogenation conditions to prepare (R)-1,2-propanediol. In the following step, the diol reacted with diethyl carbonate under suitable reaction conditions to furnish the said carbonate.

A process reported in Technical Reports, Volume 9, Number 14, page 2/7, Joint American Chemical Society, 59^{th} Northwest and 18^{th} Rocky Mountain Regional Meeting, Logan, Utah, Jun. 6-9, 2004 by Robert C. Ronald and John M. Whitaker discloses an entirely different process for the synthesis of the said intermediate compound, (R)-1,2-propylene carbonate. The synthesis used ethyl (S)-lactate as the starting material. It was transformed to an intermediate compound, (S)-2-tosyloxy-1-propanol, which, in the subsequent step, reacted with CO₂ in the presence of a phase transfer reagent, 18-crown-6, to provide the required (R)-1,2-propylene carbonate.

The process reported in the U.S. Pat. No. 5,935,946A suffers from various drawbacks. The first drawback lies in the coupling between (R)-1,2-propylene carbonate and adenine. The use of such a high boiling solvent such as N,N-dimethylformamide as the reaction medium and conducting the reaction at as high a temperature as 130° C. for a long duration of time do throw operational and work up challenges in commercial production. Secondly, the preparation of the important intermediate compound, (R)-1,2-propylene carbonate, throws a bigger challenge; its preparation when taken into account the preparation of (S)-Glycidol is cumbersome, involving many steps including a step of reductive hydrogenation using a fairly expensive heavy metal catalyst. The second process for preparing (R)-1,2-propylene carbonate as reported in Technical Report does not offer any significant advantage even though it uses an inexpensive starting material; the use of the phase transfer reagent, 18-crown-6, renders the process commercially unattractive.

### SUMMARY OF THE INVENTION

A process to prepare an intermediate of compound of Formula I used in the manufacture of anti-HIV drugs is encompassed by the present invention. The process comprises of reacting compound of Formula III with compound of Formula V. The solvent used for this reaction is selected from an alcohol, ether, water.

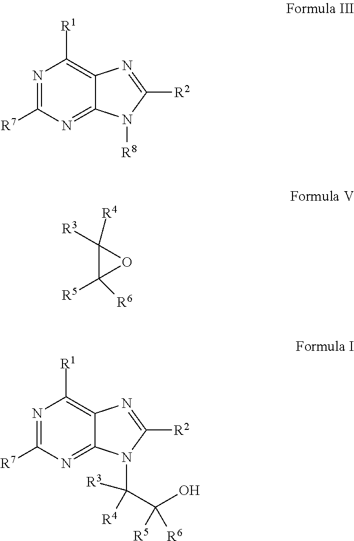

wherein,
R¹ is selected from —NH₂, Cl, Br, NHCOR″, wherein R″ is alkyl, aryl, Schiff's base of formula N═CHR′, wherein R′ is alkyl or aryl;
R² is selected from H, alkyl;
R³ and R⁴, each independently is H;
R⁵ and R⁶, each independently is H, alkyl;
R⁷ is H, alkyl; and
R⁸ is H, alkyl.

Another aspect of the present invention relates to a process for preparing compound of Formula II, used as an anti-HIV agent. The process comprises of treating compound of Formula I, prepared by the above process with compound of Formula VI in the presence of a solvent and a base, followed by hydrolysis to obtain compound of Formula II.

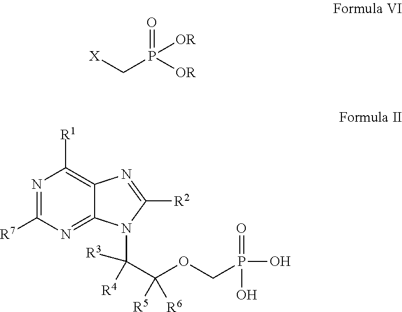

wherein,
R¹ is selected from —NH₂, Cl, Br, NHCOR″, wherein R″ is alkyl, aryl, Schiff's base of formula N═CHR′, wherein R′ is alkyl or aryl;
R² is selected from H, alkyl;
R³ and R⁴, each independently is H;
R⁵ and R⁶, each independently is H, alkyl;
R⁷ is H, alkyl;
R is alkyl such as —CH₃, C₂H₅, —CH₂CH₂—, —CH₂CH₂CH₂—; and
X is Cl, Br, I, 4-CH₃PhSO₃—, MeSO₃—, 4-CH₃PhSO₃—, substituted or unsubstituted Ar—SO₃,

### DETAILED DESCRIPTION OF THE INVENTION

According to an embodiment of the present invention a process for synthesis of compound of Formula I comprises of reacting compound of Formula III and compound of Formula V in the presence of a solvent to form compound of Formula I. The solvent is selected from an alcohol or an ether or water. The solvents used in the process are selected from tetrahydrofuran, 1,4-dioxane, monoglyme, diglyme, methyl sellosolve, methanol, ethanol, isopropanol, n-butylalcohol. The preferred solvent used in the reaction is methanol. Additionally, buffering agents are used to maintain the pH of the reaction mixture. The preferred buffering agent is NH₄Cl. The temperature is maintained in the range of 0° C. to 30° C., preferably 20° C.-23° C.

In an embodiment of the present invention, salts or derivatives of compound of Formula III are used to react with compound of Formula V. Sodium salt of compound of Formula III is preferred.

In an embodiment of the present invention, compound of Formula III on subjecting to treatment with at least one base results in the salts or derivatives thereof, which can be further utilized to prepare compound of Formula I.

The reaction scheme of synthesis of compound of Formula I is represented below:

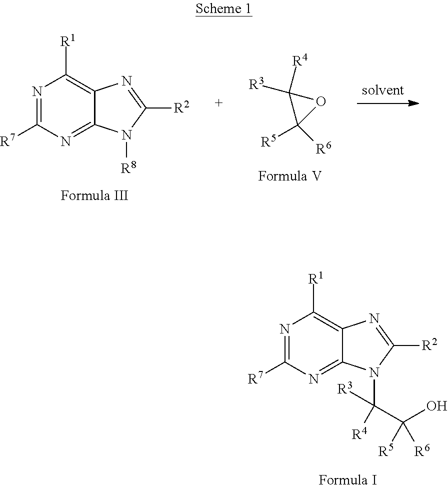

wherein,
R¹ is selected from —NH₂, Cl, Br, NHCOR″, wherein R″ is alkyl, aryl, Schiff's base of formula N═CHR′, wherein R′ is alkyl or aryl;
R² is selected from H, alkyl;
R³ and R⁴, each independently is H;
R⁵ and R⁶, each independently is H, alkyl;
R⁷ is H, alkyl; and
R⁸ is H, alkyl.

In a preferred embodiment, compound of Formula III is adenine and compound of Formula V is propylene oxide.

An embodiment of the present invention relates to a process for synthesis of 9-[2-(hydroxyl)propyl]adenine. The process comprises of reacting adenine and propylene oxide in the presence of a solvent selected from an alcohol,ether or water to form 9-[2-(hydroxyl)propyl]adenine. The solvent is selected from tetrahydrofuran, 1,4-dioxane, monoglyme, diglyme, methyl sellosolve, methanol, ethanol, isopropanol, n-butylalcohol, water. The solvent is preferably methanol. The temperature is maintained in the range of 0° C. to 30° C.

In an embodiment, sodium salt of adenine is reacted with propylene oxide.

An embodiment of the present invention relates to a process for preparing compound of Formula II. The process comprises of preparing compound of Formula I as disclosed in the aforementioned embodiment. Compound of Formula I with or without isolation is further treated with compound of Formula VI in the presence of a solvent and a base. The reaction mass on hydrolysis yields compound of Formula II.

Compound of Formula VI is

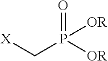

wherein,
R is alkyl such as —CH₃, C₂H₅, —CH₂CH₂—, —CH₂CH₂CH₂—;
X is Cl, Br, I, 4-CH₃PhSO₃—, MeSO₃—, 4-CH₃PhSO₃—, substituted or unsubstituted ArSO₃—

The solvent used in the reaction is dimethylformamide and the base is selected from sodium hydride, sodium amide, lithium hydride, magnesium-tert-butoxide, alkyl magnesium halide such as methyl magnesium halide, ethyl magnesium halide, isopropyl magnesium halide, tertiary butyl magnesium halide.

The reaction scheme for preparing compound of Formula II is represented below:

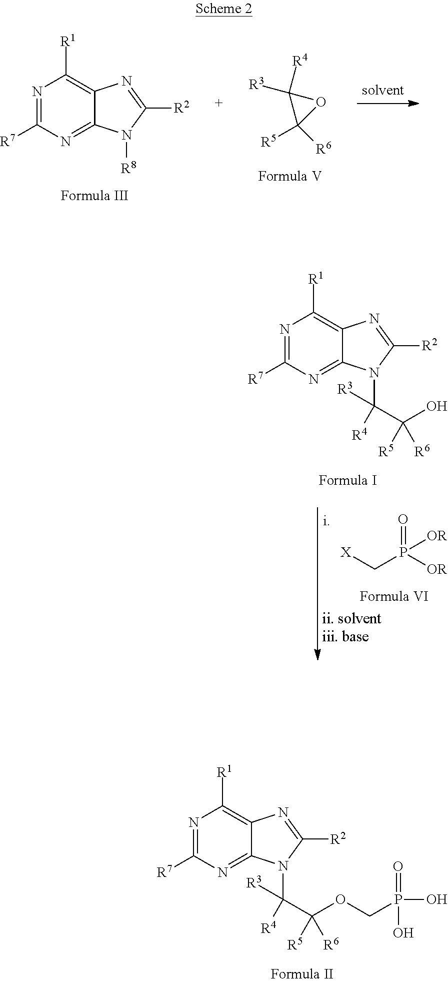

wherein,
R¹ is selected from —NH₂, Cl, Br, NHCOR″, wherein R″ is alkyl, aryl, Schiff's base of formula N═CHR′, wherein R′ is alkyl or aryl;
R² is selected from H, alkyl;
R³ and R⁴, each independently is H;
R⁵ and R⁶, each independently is H, alkyl;
R⁷ is H, alkyl;
R⁸ is H, alkyl;
R is alkyl such as —CH₃, C₂H₅, —CH₂CH₂—, —CH₂CH₂CH₂—; and
X is Cl, Br, I, 4-CH₃PhSO₃—, MeSO₃—, 4-CH₃PhSO₃—, substituted or unsubstituted ArSO₃—;

In another embodiment of the present invention, a process for synthesis of 9-[2-(R)-(phosphonomethoxy)propyl]adenine is disclosed, which comprises of preparing 9-[2-(R)-(hydroxyl)propyl]adenine by the process as disclosed in the aforesaid embodiment. 9-[2-(R)-(hydroxyl)propyl]adenine is subjected to reaction with a phosphate ester in the presence of a base and a solvent followed by hydrolysis to obtain 9-[2-(R)-(phosphonomethoxy)propyl]adenine.

9-[2-(R)-(hydroxyl)propyl]adenine with or without isolation is treated with phosphate ester. Preferably, 9-[2-(R)-(hydroxyl)propyl]adenine is not isolated and the reaction is carried out in a single pot.

The phosphate ester is

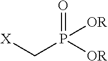

wherein,
R is alkyl such as —CH₃, C₂H₅, —CH₂CH₂—, —CH₂CH₂CH₂—; and
X is Cl, Br, I, 4-CH₃PhSO₃—, MeSO₃—, 4-CH₃PhSO₃—, substituted or unsubstituted ArSO₃—

In another embodiment of the present invention, a process to prepare compound of Formula II comprises of treating compound of Formula I with compound of Formula VI in the presence of a solvent and a base. The reaction mass on hydrolysis yields compound of Formula II. The solvent used in the reaction is dimethylformamide and the base is selected from sodium hydride, sodium amide, lithium hydride, magnesium tert-butoxide, alkyl magnesium halide such as methyl magnesium halide, ethyl magnesium halide, isopropyl magnesium halide, tertiary butyl magnesium halide.

Compound of Formula VI is

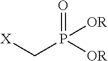

wherein,
R is alkyl such as —CH₃, C₂H₅, —CH₂CH₂—, —CH₂CH₂CH₂—;

### X is Cl, Br, I.

In a further embodiment, epoxide of Formula V can be prepared from either of the enantiomers of lactic acid, namely, L-(+)-Lactic acid or D-(−)-Lactic acid. The enantiomers of lactic acid are represented by the following structural formulae.

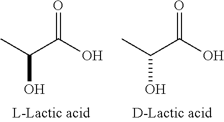

L-(+)-Lactic acid can be subjected to a sequence of reactions as demonstrated in Scheme 3 to furnish the required epoxide (V). The acid functional group of L-(+)-Lactic acid can be converted to an ester functional group by stirring a solution of L-(+)-Lactic acid in the corresponding alcohol, methanol or ethanol, with thionyl chloride or under acid catalysis, preferably sulfuric acid. The hydroxyl functional group of L-Lactate (VIII) thus obtained can be converted into a sulfonyl based leaving group, susceptible to S_{N}2 nucleophilic displacement reaction, by reacting it with a suitable sulfonyl chloride in the presence of a suitable base. The said sulfonyl chlorides can be derived from a group comprising methanesulfonyl chloride, p-toluenesulfonyl chloride, p-nitrobenzenesulfonyl chloride, p-chlorobenzenesulfonyl chloride, p-bromobenzenesulfonyl chloride or any other substituted benzenesulfonyl chloride, preferably, methanesulfonyl chloride or p-toluenesulfonyl chloride. The said base can be selected from a group of organic base comprising triethylamine, pyridine or any substituted pyridine, preferably, triethylamine.

The ester functional group of the lactate IX can be reduced to a primary hydroxyl functional group using a suitable reducing agent in a suitable solvent system. The reducing agent can be chosen from borohydride family reducing agents, for example sodium borohydride, sodium cyano borohydride or aluminum based reducing agents such as lithium aluminum hydride. The reducing agent preferred in carrying out the reduction is sodium borohydride. The appropriate reaction medium for carrying out the reduction can be a single solvent or a dual solvent system, preferably, a dual solvent system. The dual solvents can be selected from the group comprising water, water miscible solvents for example methanol, ethanol, isopropyl alcohol, tetrahydrofuran, 1,4-dioxane and chlorinated solvents for example dichloromethane, 1,2-dichloroethane etc.

The primary hydroxyl functional group of compound of formula X can be induced, under the influence of a suitable external base or the borate salts of the reaction medium itself, to displace its sulfonyloxy group in a S_{N}2 fashion whereby the chiral centre bearing the said sulfonyloxy group suffers complete reversal of its absolute configuration to furnish the epoxide of Formula V having the correct absolute configuration.

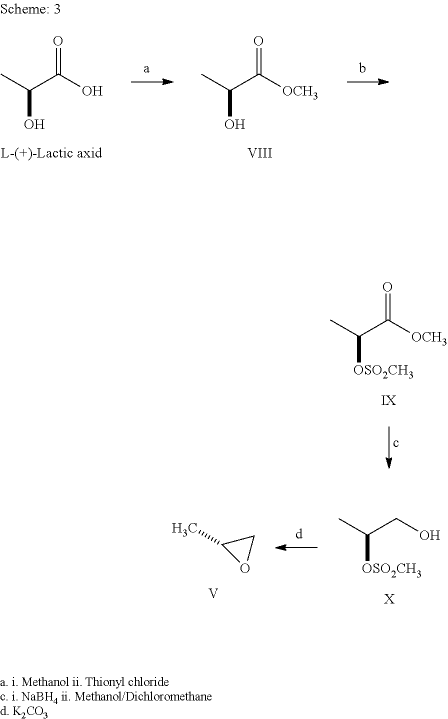

In another embodiment the ester functional group of D-lactate (XI), which can be obtained in the manner described in the preceding section, can be reduced to a primary hydroxyl functional group using a suitable reducing agent in a suitable solvent to furnish the diol (XII). The reducing agent can be selected from the borohydride group of reducing agents, preferably, sodium borohydride. The solvent can be selected from the group of alcoholic solvents, for example, methanol, ethanol, isopropyl alcohol, preferably, methanol. The primary hydroxyl group of the diol of formula (XII) obtained from the said reduction can be selectively reacted with a hindered aromatic sulfonyl chloride in the presence of a suitable base and a suitable solvent system to furnish an intermediate compound of formula (XIII). The secondary hydroxyl functional group of the said intermediate compound can be induced, under the influence of a suitable base, to displace its aromatic sulfonyloxy group in a S_{N}2 fashion to furnish the epoxide of Formula V having the correct absolute configuration. The absolute configuration of the chiral centre bearing the secondary hydroxyl of alcohol compound of formula XII is retained during the displacement reaction.

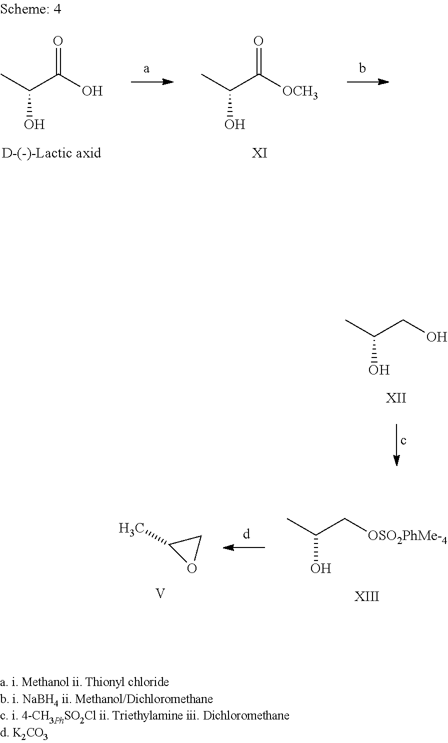

In further embodiments, compound of Formula I and Formula II can be used to prepare tenofovir alafenamide fumarate and tenofovir disoproxyl fumarate, which are used as anti-HIV agents.

Compound of Formula I is an important intermediate in the synthesis of the crucial anti-HIV precursor of Formula II. The process as covered by the aforesaid embodiments uses inexpensive and readily available compounds. L-(+)-lactic acid is used as the starting material which is converted to the product following a straightforward pathway. The present invention has substantial advantage in terms of the ease of industrial operation and cost over the other processes in industrial preparation of tenofovir, the key intermediate in tenofovir based anti-HIVdrugs. The invention facilitates preparation of compound of Formula II, particularly 9-[2-(R)-(phosphonomethoxy)propyl]adenine in a single pot operation.

The following examples illustrate the invention but are not limiting thereof:

### Example: 1 Preparation of Methyl L-(+)-lactate (VIII)

Thionyl chloride (96.72 ml) was added drop by drop to a solution of L-(+) lactic acid (100 g) in methanol (500 ml) at −10° C. over a period of 1 h. After the completion of addition, the reaction mixture was warmed to room temperature and stirred at this temperature until the reaction was complete as indicated by TLC. The reaction mixture was concentrated at 90° C. to furnish an oil which was dissolved in dichloromethane (250 ml). The organic layer was washed with saturated sodium bicarbonate solution (2×150 ml), dried over sodium sulfate and concentrated at 50° C. to give VIII; yield: 79 g.

### Example: 2 Preparation of Methyl 2-(S)-methylsulfonyloxypropionate (IX)

Triethylamine (107 ml) was added to a cold, stirred solution of methyl (S)-lactate (40 g) in dichloromethane (400 ml) −10° C. Methanesulfonyl chloride (38.8 ml) was added to the reaction mixture drop by drop over a period of 1 h at this temperature, and the reaction mixture was stirred at this temperature for about 2 h when the reaction was complete, as indicated by TLC. The reaction mixture was poured on cold water (400 ml) and stirred for 10 minutes. The separated organic layer was washed with 5% dilute hydrochloric acid (1×80 ml), saturated sodium bicarbonate solution (1×100 ml), brine (1×100 ml), dried over sodium sulfate and concentrated at 50° C. to furnish the desired product; 48.2 g.

### Example: 3 Preparation of 2-(R)-(+)-propylene Oxide (V)

### Method: A

Methanol (6 ml) was added to a solution of the mesylated methyl (S)-lactate (3 g) in dichloromethane (9 ml). The reaction mixture was cooled to −10° C. and sodium borohydride (748 mg) was added to the mixture in divided portions over a period of 0.5 h at this temperature. After 4 h at this temperature, when the reaction was complete as indicated by TLC, the reaction mixture was diluted with water (10 ml) and dichloromethane (20 ml) and warmed to room temperature. After 10 minutes at room temperature, layers were separated. The organic layer was washed with 10% aqueous citric acid solution (1×10 ml), brine (1×10 ml), dried over sodium sulfate and concentrated under reduced pressure at 40° C. to give mesylated alcohol; yield: 1.1 g. The crude mesylated alcohol was treated with potassium carbonate, and distilled to furnish (R)-(+)-propylene oxide.

### Example: 4 Preparation of 2-(R)-hydroxypropanol (XII)

Sodium borohydride (1.81 g) was added to a stirred, cold solution of methyl (D)-lactate (5 g) in methanol (25 ml) at −5° C. in divided portions over a period of 0.5 h. The reaction mixture was stirred at this temperature for 3 h when the reaction was complete as indicated by TLC. The reaction mixture was neutralized with concentrated hydrochloric acid until the pH of the mixture attained the range 6-7, and then concentrated under reduced pressure at 40° C. The residue thus obtained was dissolved in ethyl acetate (30 ml) and filtered. The filtrate was washed with water (1×20 ml), brine (1×5 ml), dried over sodium sulfate and concentrated under reduced pressure at 40° C. to give the product XII; yield: 1.97 g.

### Example: 5 Preparation of 2-(R)-(+)-propylene Oxide (V)

### Method: B

Pyridine (1.58 ml) was added to a solution of the diol (XII) (0.5 g) in dichloromethane (6 ml) at room temperature. 4-Tolunesulfonyl chloride (1.5 g) was added at −78° C. and the reaction mixture was allowed to warm to room temperature. The reaction mixture was stirred at this temperature until the reaction was complete as indicated by TLC. The reaction mixture was poured into cold water to separate the layers. The organic layer was washed with 10% dilute hydrochloric acid, water, brine, dried over sodium sulfate and concentrated at 40° C. to furnish a residue which was purified to furnish the said compound XIII; yield: 0.7 g. The alcohol was was treated with potassium carbonate, and distilled to furnish (R)-(+)-propylene oxide.

### Example: 6 Preparation of 9-[2-(R)-(Hydroxy)propyl]adenine (I)

### Method-A

Sodium hydroxide (3 g) was added to a stirred suspension of adenine (10 g) in water (50 ml) at room temperature, and the reaction mixture was heated to 100-110° C. After 5 hrs at this temperature, the reaction mixture was allowed to cool down to 25° C. Ammonium chloride (3.96 g) and 2-(R)-(+) propylene oxide (6.44 g) were added in succession to the reaction mixture at this temperature. The reaction mixture was maintained at this temperature until the reaction was complete as indicated by TLC. The reaction mixture was concentrated under reduced pressure at 55° C. to furnish the desired product, contaminated with unidentified polar impurities. The crude material was purified by column chromatography using a solvent system comprising of methanol and dichloromethane (1:9) as the eluant to furnish 9-[2-(R)-(hydroxyl)propyl]adenine as a white solid; yield: 5.5 g.

### Example: 7 Preparation of 9-[2-(R)-(Hydroxy)propyl]adenine (I)

### Method-B

Potassium tert-butoxide (4.15 g) was added to a stirred suspension of adenine (10 g) in methanol (150 ml) at 0° C. The reaction mixture was allowed to warm to room temperature, and stirred at this temperature for 30 minutes. R-(+)-Propylene oxide (6.44 g) was added and the reaction mixture was stirred at this temperature until the reaction was complete, as indicated by TLC. The reaction mixture was neutralized with methanesulfonic acid and filtered. The filtrate was concentrated under reduced pressure to furnish the desired product, contaminated with unidentified polar impurities. The crude material was purified by column chromatography using a solvent system comprising of methanol and dichloromethane (1:9) as the eluant to furnish 9-[2-(R)-(hydroxyl)propyl]adenine as a white solid; yield: 6.2 g.

### Example: 8 Preparation of 9-[2-(R)-(Hydroxyl)propyl]adenine (I)

### Method-C

Potassium carbonate (204 mg) was added to a stirred suspension of adenine (5 g) in a solvent mixture comprising of methanol (13 ml) and N,N-dimethylformamide (2.5 ml) at 0° C. The reaction mixture was allowed to warm to room temperature. (R)-(+)-Propylene oxide (3.78 ml) was added to the reaction mixture in three divided batches over a period of 20 h, and the reaction mixture was stirred at this temperature until the reaction was complete, as indicated by TLC. The reaction mixture was neutralized with methanesulfonic acid and concentrated under reduced pressure. Toluene (15 ml) was added to the residue thus obtained. The reaction mixture was cooled to 0° C. and stirred at this temperature for 2 h to maximize the precipitation of the desired compound. The precipitated material was filtered and dried to furnish 9-[2-(R)-(hydroxyl)propyl]adenine, crude yield: 6.8 g. The product was contaminated with unidentified polar impurities. The crude material was carried over to the following step without any purification.

### Example: 9 Preparation of 9-[2-(R)-(hydroxyl)propyl]adenine (I)

### Method-D

Magnesium di-tert-butoxide (630 mg) was added to a stirred suspension of adenine (5 g) in a solvent mixture comprising of methanol (15 ml) and N,N-dimethylformamide (15 ml) at 0° C. The reaction mixture was warmed to room temperature. (R)-(+)-Propylene oxide (3.78 ml) was added to the reaction mixture in three divided batches over a period of 20 h and the mixture was stirred at room temperature until the reaction was complete, as indicated by TLC. The reaction mixture was then neutralized with methanesulfonic acid and concentrated under reduced pressure. Toluene (15 ml) was added to the residue thus obtained. The reaction mixture was cooled to 0° C. and stirred at this temperature for 2 h to maximize the precipitation of the desired compound. The precipitated material was filtered and dried to furnish 9-[2-(R)-(hydroxyl)propyl]adenine, crude yield: 6.4 g. The product was contaminated with unidentified polar impurities. The crude material was carried over to the following step without any purification.

### Example: 10 Preparation of 9-[2-(R)-(Hydroxyl)propyl]adenine (I)

### Method: E

Magnesium di-tert-butoxide (100 mg) was added to a stirred suspension of adenine (2 g) in methanol (12 ml) at 0° C. After 6 h at this temperature, sodium hydroxide (24 mg) and (R)-(+)Propylene oxide (1.25 g) were added in succession to the reaction mixture. The mixture was allowed to warm to room temperature, stirred at this temperature until the reaction was complete as indicated by TLC and concentrated under reduced pressure. Toluene (6 ml) was added to the residue thus obtained. The reaction mixture was cooled to 0° C. and stirred at this temperature for 2 h to maximize the precipitation of the desired compound. The precipitated material was filtered and dried to furnish 9-[2-(R)-(hydroxyl)propyl]adenine, crude yield: 3.2 g. The product was contaminated with unidentified polar impurities. The crude material was carried over to the following step without any purification.

### Example: 11 Preparation of 9-[2-(R)-(phosphonomethoxy)propyl]adenine (II)

### Method-A

Magnesium di-tert-butoxide (51 mg) was added to a stirred suspension of adenine (2 g) in methanol (12 ml) at 10° C. After 4 h at this temperature, sodium hydroxide (12 mg) and (R)-(+)-propylene oxide (1.25 g) were added in succession to the reaction mixture. The reaction mixture was allowed to warm to room temperature and stirred at this temperature until the reaction was complete, as indicated by TLC. Methanol was evaporated under vacuum. N, N-Dimethylformamide (16 ml) was added to the crude reaction mixture thus obtained and the mixture was heated to 60-70° C. Magnesium di-tert-butoxide (7 g) was added to the reaction mixture in four divided batches over a period of 15 minutes at this temperature. The mixture was heated to 80-90° C., and stirred at this temperature for 30 minutes. Diethyl p-toluenesulfonyloxymethylphosphonate (13.2 g) was added drop by drop to the reaction mixture over a period of 4 h, and the mixture was stirred at this temperature until the reaction was complete, as indicated by TLC. N,N-Dimethylformamide was distilled out under vacuum at 90-100° C. Aqueous hydrobromic acid (48% w/w, 30 ml) was then added to the residue and the reaction mixture was heated to gentle reflux. After approximately 20 h at this condition, the reaction mixture was allowed to cool down to room temperature and filtered. The filtered solid was washed with dichloromethane (10 ml). The washing was concentrated to furnish a residue. The residue was combined with the filtrate and the combined filtrate was washed with dichloromethane (2×10 ml). To the aqueous layer, an aqueous solution of sodium hydroxide (50%) was added until the pH attained 2.1-3. After several hours at room temperature, the aqueous layer was cooled to 0-5° C. and stirred at this temperature for further hours to maximize precipitation of the desired product from the solution. The precipitated solid was filtered, washed with cold water (1×5 ml), acetone (2×5 ml) and dried to obtain 9-[2-(R)-(phosphonomethoxy)propyl]adenine; yield: 1.42 g.

### Example: 12 Preparation of 9-[2-(R)-(phosphonomethoxy)propyl]adenine (II)

### Method-B

Magnesium di-tert-butoxide (1.76 g) was added in four divided batches to a stirred suspension of 9-[2-(R)-(hydroxyl)propyl)adenine] (1 g) in N,N-dimethylformamide (3 ml) at 60-70° C. over a period of 1 h. The reaction mixture was heated to 90° C. and diethyl chloromethylphosphonate (1.93 g) was added drop by drop to the reaction mixture over a period of 4 h. The reaction mixture was maintained at this temperature until the reaction was complete, as indicated by TLC. N,N-Dimethylformamide was distilled out under vacuum at 90-100° C. To the residue thus obtained, aqueous hydrobromic acid (48% w/w, 10 ml) was added and the reaction mixture was heated to gentle reflux. After approximately 20 h at this condition, the reaction mixture was allowed to cool down to room temperature and filtered. The filtered solid was washed with dichloromethane (10 ml). The washing was concentrated to furnish a residue. The residue was combined with the filtrate and the combined filtrate was washed with dichloromethane (2×10 ml). To the aqueous layer, an aqueous solution of sodium hydroxide (50%) was added until the pH attained 2.1-3. After several hours at room temperature, the aqueous layer was cooled to 0-5° C. and stirred at this temperature for further hours to maximize precipitation of the desired product from the solution. The precipitated solid was filtered, washed with cold water (1×5 ml), acetone (2×5 ml) and dried to obtain 9-[2-(R)-(phosphonomethoxy)propyl]adenine; yield: 0.7 g.

### Example: 13 Preparation of diethyl (chloromethyl)phosphonate (VI)

Pyridine (108 ml) was added to a stirred solution of (hydroxymethyl)phosphonic acid (50 g) in benzene (300 ml) at room temperature. The reaction mixture was heated to 60° C. Thionyl chloride (100 ml) was added to the mixture over a period of 30 min at this temperature. After 1 h at this temperature, the reaction mixture was allowed to cool down to room temperature, stirred for 3 h at this temperature and cooled to 10° C. The precipitated solid was filtered and washed with benzene (100 ml). The washing was combined with the filtrate and the combined filtrate was concentrated at 100° C. to furnish an oil. The oil was fraction distilled under reduced pressure to furnish a colourless liquid; yield: 22 g. The liquid was cooled 0° C. Ethanol (40 ml) was added to the liquid at this temperature. The reaction mixture was allowed to warm to room temperature, stirred for 3 h at this temperature and diluted with dichloromethane (100 ml). The combined organic layer was washed with water (2×50 ml), brine (50 ml), dried over sodium sulfate and concentrated under reduced pressure to furnish diethyl (chloromethyl)phosphonate as a colorless liquid; yield: 18 g.

1. A process for synthesis of compound of Formula I, the process comprising
reacting compound of Formula III with compound of Formula V in the presence of a solvent selected from an alcohol, ether, water to form compound of Formula I. 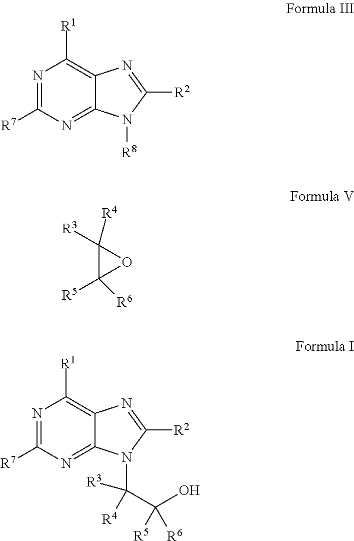
wherein,
R¹ is selected from —NH₂, Cl, Br, NHCOR″, wherein R″ is alkyl, aryl, Schiff's base of formula N═CHR′, wherein R′ is alkyl or aryl;
R² is selected from H, alkyl;
R³ and R⁴, each independently is H;
R⁵ and R⁶, each independently is H, alkyl;
R⁷ is H, alkyl; and
R⁸ is H, alkyl.

2. A process for synthesis of compound of Formula II, the process comprising
reacting compound of Formula III with compound of Formula V in the presence of a solvent selected from an alcohol, ether, water to form compound of Formula I; and
treating compound of Formula I with compound of Formula VI in the presence of a solvent and a base followed by hydrolysis to obtain compound of Formula II. 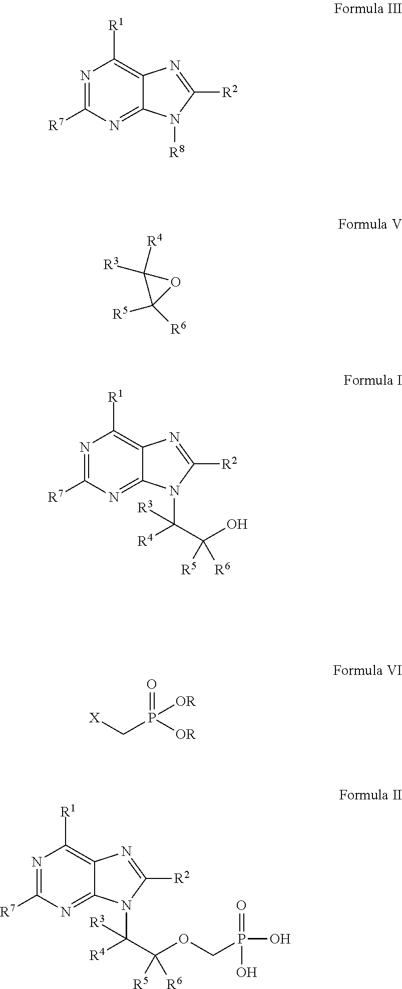
wherein,
R¹ is selected from —NH₂, Cl, Br, NHCOR″, wherein R″ is alkyl, aryl, Schiff's base of formula N═CHR′, wherein R′ is alkyl or aryl;
R² is selected from H, alkyl;
R³ and R⁴, each independently is H;
R⁵ and R⁶, each independently is H, alkyl;
R⁷ is H, alkyl;
R⁸ is H, alkyl; and
X is Cl, Br, I, 4-CH₃PhSO₃—, MeSO₃—, 4-CH₃PhSO₃—, substituted or unsubstituted ArSO₃—.

3. The process as claimed in claim 1, wherein
R¹ is —NH₂;
R², R³, R⁴, R⁵, R⁷, R⁸ each independently is H; and
R⁶ is CH₃.

4. The process as claimed in claim 1, wherein salts or derivatives of compound of Formula III are reacted with compound of Formula V.

5. The process as claimed in claim 1, comprising adding a buffering agent selected from NH₄Cl in the step of forming compound of Formula I.

6. The process as claimed in claim 1, wherein the solvent used to form compound of Formula I is selected from tetrahydrofuran, 1,4-dioxane, monoglyme, diglyme, methyl sellosolve, methanol, ethanol, isopropanol, n-butylalcohol, water.

7. The process as claimed in claim 2, wherein the solvent used to form compound of Formula II from compound of Formula I and compound of Formula VI is dimethylformamide and the base is selected from sodium hydride, sodium amide, lithium hydride, magnesium tert-butoxide, alkyl magnesium halide such as methyl magnesium halide, ethyl magnesium halide, isopropyl magnesium halide, tertiary butyl magnesium halide.

8. The process as claimed in claim 1, wherein compound of Formula I is formed at a temperature in the range of 0° C. to 30° C.

9. The process as claimed in claim 1, wherein compound of Formula V is prepared from L-(+) Lactic acid represented by

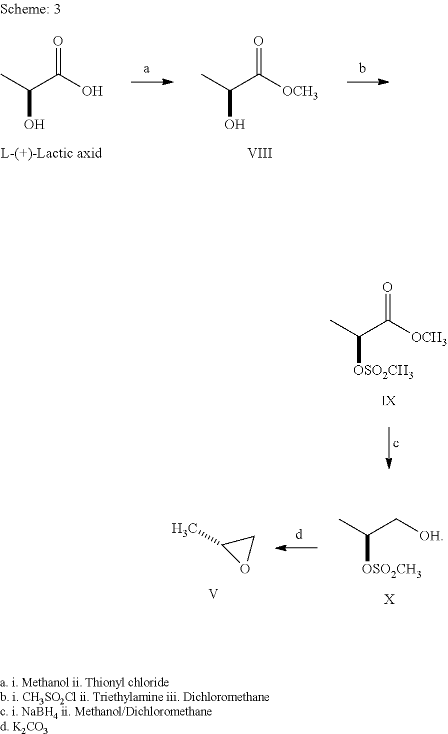

10. The process as claimed in claim 1, wherein compound of Formula V is prepared from D-(−) Lactic acid represented by

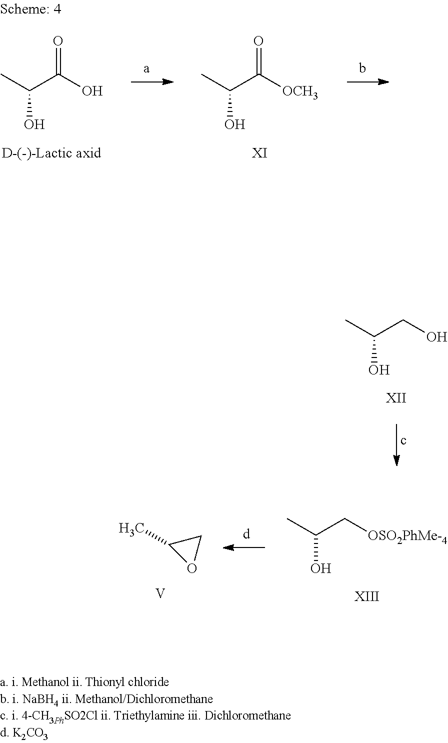

11. A process for synthesis of 9-[2-(hydroxyl)propyl]adenine, the process comprising reacting adenine and propylene oxide in the presence of a solvent selected from an alcohol, ether, water to form 9-[2-(hydroxyl)propyl]adenine.

12. The process as claimed in claim 11, wherein salts or derivatives of adenine are reacted with propylene oxide, preferably sodium salt of adenine is reacted with propylene oxide.

13. The process as claimed in claim 11, wherein the solvent is selected from tetrahydrofuran, 1, 4-dioxane, monoglyme, diglyme, methyl sellosolve, methanol, ethanol, isopropanol, n-butylalcohol, water.

14. The process as claimed in claim 11, wherein the process is carried out in the presence of a buffering agent selected from NH₄Cl.

15. A process for synthesis of 9-[2-phosphonomethoxypropyl]adenine, the process comprising of
reacting adenine with propylene oxide in the presence of a solvent selected from an alcohol, ether, water to form 9-[2-(hydroxyl)propyl]adenine; and
treating 9-[2-(hydroxyl)propyl]adenine with a phosphate ester in the presence of a solvent and a base followed by hydrolysis to obtain 9-[2-phosphonomethoxypropyl]adenine.

16. The process as claimed in claim 15, wherein the phosphate ester is 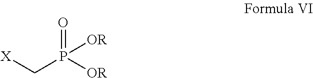
wherein,
R is alkyl such as —CH₃, C₂H₅, —CH₂CH₂—, —CH₂CH₂CH₂—; and
X is Cl, Br, I, 4-CH₃PhSO₃—, MeSO₃—, 4-CH₃PhSO₃—, substituted or unsubstituted ArSO₃—.

17. The process as claimed in claim 15, wherein the solvent used to prepare [2-(hydroxyl)propyl]adenine is selected from tetrahydrofuran, 1, 4-dioxane, monoglyme, diglyme, methyl sellosolve, methanol, ethanol, isopropanol, n-butylalcohol, water.

18. The process as claimed in claim 15, wherein the solvent used to obtain 9-[2-(R)-phosphonomethoxypropyl]adenine is dimethylformamide and the base is selected from sodium hydride, sodium amide, lithium hydride, magnesium tert-butoxide, alkyl magnesium halide such as methyl magnesium halide, ethyl magnesium halide, isopropyl magnesium halide, tertiary butyl magnesium halide.

19. A process for synthesis of 9-[2-phosphonomethoxypropyl]adenine, the process comprising of
treating 9-[2-(hydroxyl)propyl]adenine with a phosphate ester of Formula VI in the presence of a solvent and a base followed by hydrolysis to obtain 9-[2-phosphonomethoxypropyl]adenine. 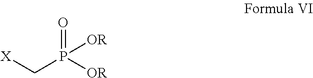
wherein,
R is alkyl such as —CH₃, C₂H₅, —CH₂CH₂—, —CH₂CH₂CH₂—; and
X is Cl, Br, I.

20. The process as claimed in claim 1, wherein the process is carried out in a single pot operation.
